(19) United States
(12) Patent Application Publication
(10) Pub. No.: US 2022/0253687 A1
(43) Pub. Date: Aug. 11, 2022

(54) GENERIC DISCRIMINATIVE INFERENCE WITH GENERATIVE MODELS

(71) Applicant: INTERNATIONAL BUSINESS MACHINES CORPORATION, Armonk, NY (US)

(72) Inventors: Kohei Miyaguchi, Tokyo (JP); Takayuki Katsuki, Tokyo (JP); Akira Koseki, Yokohama-shi (JP); Toshiya Iwamori, Tokyo (JP)

(21) Appl. No.: 17/155,335

(22) Filed: Jan. 22, 2021

(51) Int. Cl. G06N 3/08; G06N 3/04; G06N 7/00

(52) U.S. Cl. CPC G06N 3/08; G16H 10/60; G06N 7/005; G06N 3/0454

(57) ABSTRACT

A computer-implemented method for computing an objective function of discriminative inference with generative models with incomplete data in which some of entries are missing is provided including acquiring an incomplete set of covariates x̅ including incomplete features \tilde{x} and an incomplete pattern m indicating missing entries of the incomplete features \tilde{x} and computing a predictive distribution p_{θ}(y|x̅) of an outcome y by using the incomplete set of covariates x̅ and a parameter θ, the parameter θ being unknown. Learning of the parameter θ is performed by minimizing an objective function [symbol] (θ):=−ln p_{θ}(y|x̅)=ln p_{θ}(\tilde{x}|m)−ln p_{θ}(y,x̅|m), and the objective function [symbol] (θ) is bounded with a difference between a marginal evidence upper bound [symbol] _{MEUBO} and a joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

### BACKGROUND

The present invention relates generally to learning with incomplete data, and more specifically, to a method for computing an objective function of discriminative inference with generative models with incomplete data in which some of entries are missing.

Electronic health records (EHRs) present a wealth of data that are vital for improving patient-centered outcomes, although the data can present significant statistical challenges. In particular, EHR data includes substantial missing information that if left unaddressed could reduce the validity of conclusions drawn. Properly addressing the missing data issue in EHR data is complicated by the fact that it is sometimes difficult to differentiate between missing data and a negative value. For example, a patient without a documented history of heart failure may truly not have disease or the clinician may have simply not documented the condition.

Generative modeling is known to be useful in this context because such generative modeling can learn predictive distributions of survival times and can handle missing values. However, it is also known that training generative models with respect to the pure objective of prediction can be intractable if the models are complex.

### SUMMARY

In accordance with an embodiment, a computer-implemented method for computing an objective function of discriminative inference with generative models with incomplete data in which some of entries are missing is provided. The computer-implemented method includes acquiring an incomplete set of covariates x̅ including incomplete features \tilde{x} and an incomplete pattern m indicating missing entries of the incomplete features \tilde{x} and computing a predictive distribution p_{θ}(y|x̅) of an outcome y by using the incomplete set of covariates x̅ and a parameter θ, the parameter θ being unknown, wherein learning of the parameter θ is performed by minimizing an objective function [symbol] (θ):=−ln p_{θ}(y|x̅)=ln p_{θ}(\tilde{x}|m)−ln p_{θ}(y,\tilde{x}|m), and the objective function [symbol] (θ) is bounded with a difference between a marginal evidence upper bound [symbol] _{MEUBO} and a joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

In accordance with another embodiment, a computer program product for computing an objective function of discriminative inference with generative models with incomplete data in which some of entries are missing is provided. The computer program product includes a computer readable storage medium having program instructions embodied therewith, the program instructions executable by a computer to cause the computer to acquire an incomplete set of covariates x̅ including incomplete features \tilde{x} and an incomplete pattern m indicating missing entries of the incomplete features \tilde{x} and compute a predictive distribution p_{θ}(y|x̅) of an outcome y by using the incomplete set of covariates x̅ and a parameter θ, the parameter θ being unknown, wherein learning of the parameter θ is performed by minimizing an objective function [symbol] (θ):=ln p_{θ}(y|x̅)=ln p_{θ}(\tilde{x}|m)−ln p_{θ}(y,\tilde{x}|m), and the objective function [symbol] (θ) is bounded with a difference between a marginal evidence upper bound [symbol] _{MEUBO} and a joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

In accordance with yet another embodiment, a computer-implemented method for computing an objective function of discriminative inference with generative models with incomplete data in which some of entries are missing is provided. The computer-implemented method includes combining a plurality of probability models with a discriminative variational autoencoder (DVAE), computing a joint evidence lower bound [symbol] _{JELBO} via a first set of the one or more of the plurality of probability models, and computing a marginal evidence upper bound [symbol] _{MEUBO} via a second set of the one or more of the plurality of probability models.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention will provide details in the following description of preferred embodiments with reference to the following figures wherein:

FIG. 1 shows an exemplary discriminative variational autoencoder (DVAE), in accordance with an embodiment of the present invention;

FIG. 2 is an exemplary computation graph of the DVAE of FIG. 1, in accordance with an embodiment of the present invention;

FIG. 3 is a block/flow diagram of an exemplary method for computing an objective function of discriminative inference with generative models with incomplete data, in accordance with an embodiment of the present invention;

FIG. 4 illustrates joint evidence lower bound and marginal evidence upper bound equations, in accordance with an embodiment of the present invention;

FIG. 5 is an exemplary algorithm for training the DVAE, in accordance with an embodiment of the present invention;

FIG. 6 is an exemplary neuromorphic and synaptronic network including a crossbar of electronic synapses interconnecting electronic neurons and axons, in accordance with an embodiment of the present invention;

FIG. 7 is a block diagram of components of a computing system including a computing device employing the algorithm of FIG. 5 for training the DVAE via an artificial intelligence (AI) accelerator chip, in accordance with an embodiment of the present invention;

FIG. 8 is a block/flow diagram of an exemplary cloud computing environment, in accordance with an embodiment of the present invention;

FIG. 9 is a schematic diagram of exemplary abstraction model layers, in accordance with an embodiment of the present invention;

FIG. 10 illustrates practical applications for employing the DVAE via an AI accelerator chip, in accordance with an embodiment of the present invention; and

FIG. 11 is a block/flow diagram of a practical application including health care records for employing the DVAE via the AI accelerator chip, in accordance with an embodiment of the present invention.

Throughout the drawings, same or similar reference numerals represent the same or similar elements.

### DETAILED DESCRIPTION

Embodiments in accordance with the present invention provide methods and devices for advantageously learning with incomplete data by employing a generic method for discriminative training of generative models. The exemplary methods utilize blackbox variational inference frameworks so that the methods can be applied to a variational autoencoder, which is a state-of-the-art generative model, while fully making use of standard automatic differentiation libraries.

The exemplary methods are concerned with the issue of learning with incomplete data, which often arises in real-life data due to the lack of data collecting resources. In particular, electronic health records (EHR) is an example of such datasets. There are various types of data to describe patients such as demographic characteristics, medical measurement data obtained with various instruments and historical collection of those, while most of them are not necessarily available with all the patients due to limited data collecting resources, non-standardized medical equipment and legal and/or privacy concerns.

With the application to EHRs in mind, the exemplary embodiments consider the problem of survival time prediction, formalized as follows. Each input is an incomplete set of covariates (patient characteristics and measurements) represented with a feature-and-mask pair x̅=(\tilde{x},m) such that \tilde{x}∈ [symbol] ^{d} are the incomplete covariate features encoded in a real vector and m∈{0,1}^{d} indicates the missing entries of \tilde{x}: For all j∈[d], mⱼ=1, if and only if, \tilde{x}ⱼ is missing and in that case filled with zero, \tilde{x}ⱼ=0, and otherwise the true values \tilde{x}ⱼ=xⱼ are revealed.

Given the incomplete covariates x̅, the exemplary embodiments predict the outcome (e.g., the survival time of the patient) y∈ [symbol] , which is modeled with a predictive distribution y˜p_{θ}(y|x̅). Here, the parameter θ is unknown and should be learned from data.

One straightforward way to deal with such incompletion is to follow a two-step approach, that is, first learn generative models of covariates p_{θ}(x), and then utilize the learnt information in the subsequent prediction, e.g., imputing the missing values with Monte-Carlo samples \hat{x}˜p_{θ}(x|x̅) and then passing it as the input of prediction, p_{θ}(y|\hat{x}).

This method has an advantage that the predictive performance is enhanced with the knowledge on the distribution of x. On the other hand, it is recognized that discriminative models that directly model the conditional density p_{θ}(y|x̅) exhibit superior performance in some cases, which is in accordance with the general tendency known outside the realm of covariate incompletion.

In particular, the exemplary embodiments decompose the model as p_{θ}(y,x̅)=p_{θ}(y|x̅) p_{θ}(x̅) and replace the latter parameter with a dummy one, p_{θ,θ′}(y,x̅)=p_{θ}(y|x̅) p_{θ},(x̅), where p_{θ}(y|x̅)=p_{θ}(y,x̅)/p_{θ}(x̅).

This decomposition splits the evidence of the total generative model into a purely generative and purely discriminative terms:

$$ln {p}_{θ , {θ}^{′}} ( y , \overline{x} ) = ln {p}_{θ} ( y \vert \overline{x} ) + ln {p}_{θ} , ( \overline{x} ) ,$$

where the first term ln p_{θ}(y|x̅) is discriminative and the second term ln p_{θ},(x̅) is generative, and whose maximization is equivalent to the discriminative training from the viewpoint of θ. Here, the difference with the purely discriminative methods is noted as follows. The exemplary embodiments of the present invention explicitly model the generative process of y and x̅ and then compute the conditional density via the Bayes rule. This way, the exemplary methods can explicitly incorporate the knowledge of generative distribution without introducing any non-discriminative terms to the objective function, unlike the two-step methods. This inference scheme is referred to as the discriminative inference with generative models (DIGM).

Although this approach is theoretically well-founded and seems promising, it is known that the transformation from the joint density p_{θ}(y,x̅) to the conditional density p_{θ}(y|x̅) is computationally demanding, especially in the presence of covariate incompletion.

The difficulty is best seen from the fact that the objective function includes two computationally expensive integrals, that is:

$$L ( θ ) : = - ln {p}_{θ} ( y \vert \overline{x} ) = ln {p}_{θ} ( \tilde{x} \vert m ) - ln {p}_{θ} ( y , \tilde{x} \vert m ) = ln \int {p}_{θ} ( y , x \vert m ) {dydx}_{\{ j : {m}_{j} = 1 \}} ln \int {p}_{θ} ( y , x \vert m ) {dx}_{\{ j : {m}_{j} = 1 \}}$$

where the variables under the integrals vary in accordance with the mask vector m. This explains why the application of DIGM to the context of covariate incompletion has been limited to some special cases such as partially observed exponential families and Gaussian processes.

The exemplary embodiments of the present invention address the issue of the applicability of DIGM under covariate incompletion by constructing a generic approximation of the above equation. More specifically, the exemplary embodiments introduce a new black-box approximation of the integrals, which opens up the possibility of much greater degree of freedom in the choice of generative models. Additionally, the exemplary embodiments utilize the proposed approximation and present a variant of variational autoencoders (VAE) designed for performing DIGM with neural networks.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

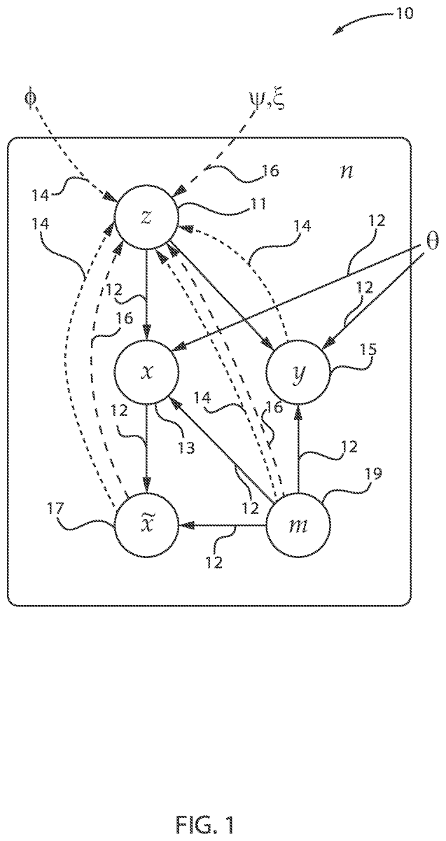

FIG. 1 shows an exemplary discriminative variational autoencoder (DVAE), in accordance with an embodiment of the present invention.

The exemplary embodiments present the discriminative variational autoencoder (DVAE), which performs DIGM with incomplete covariates. DVAE includes a generative network, two variational networks, and a surrogate network as shown in FIG. 1.

Regarding the generative probabilistic model, the exemplary embodiments suppose that the joint probability density of (y, x) given m is modeled with a generative neural network θ. That is, with some latent noise variable z and the corresponding prior p(z),

$${p}_{θ} ( y , \tilde{x} \vert m ) = \int p ( z ) {p}_{θ} ( y , x \vert z , m ) p ( \tilde{x} \vert x , m ) dxdz$$

where x∈ [symbol] ^{d} denotes the complete covariates, p_{θ}(y,x|z):=p(y,x|θ(z)) is the density given by the neural network θ, and

$$p ( \tilde{x} \vert x , m ) := {\prod }_{j = 1}^{d} δ ( {\tilde{x}}_{j} - ( 1 - {m}_{j} ) {x}_{j} )$$

is corresponding to the masking process. Here, δ denotes Dirac's delta function. It is noted that the exemplary embodiments treat m as a conditional since there is no interest in the distribution of missing patterns. For simplicity, the exemplary embodiments further limit the scope to where the individual covariates xⱼ and the target y are mutually conditionally independent given z and m, which allows the exemplary methods to efficiently compute some of marginal distributions,

$${p}_{θ} ( y , \tilde{x} \vert z , m ) = {p}_{θ} ( y \vert z , m ) {\prod }_{j : {m}_{j} = 0} {p}_{θ} ( {x}_{j} = {\tilde{x}}_{j} \vert z , m ) ,$$

$${p}_{θ} ( \tilde{x} \vert z , m ) = {\prod }_{j : {m}_{j} = 0} {p}_{θ} ( {x}_{j} = {\tilde{x}}_{j} \vert z , m ) .$$

Regarding discriminative interference, the goal of DIGM is to minimize the conditional negative log-likelihood with respect to some training data [symbol] :={(yⁱ,x̅ⁱ)}_{i∈[n]}, given by [symbol] ₙ(θ):=Σᵢ₌₁ⁿ [symbol] ⁱ(θ), where [symbol] ⁱ(θ):=−ln p_{θ}(yⁱ,x̅ⁱ) denotes the individual loss of the i-th instance (yⁱ,x̅ⁱ)∈ [symbol] × [symbol] ^{d}, corresponding to a patient in the dataset.

In the following, the exemplary methods introduce an approximation of the individual losses [symbol] ⁱ (θ) and the exemplary methods omit the patient index i for the ease of exposition.

Now, it is observed that [symbol] (θ)=ln p_{θ}(y,x̅|m)+ln p_{θ}(x̅|m).

Since both terms in the right-hand side include intractable integrals inside, the exemplary methods resort to the variational inference framework. In particular, the exemplary methods bound [symbol] (θ) from above with a quantity that is computationally tractable and readily usable with gradient-based optimization algorithms. To this end, the exemplary methods introduce three neural networks φ, ψ, and ξ which are trained together with the generative network θ.

Regarding the Joint Evidence Lower Bound (JELBO), the first term is bounded with the standard variational lower bound with a negative sign on both sides,

$$- ln {p}_{θ} ( y , \tilde{x} \vert m )$$

$$\leq - ln {p}_{θ} ( y , \tilde{x} \vert m ) + {D}_{K L} ( {q}_{ϕ} ( Z \vert y , \overline{x} ) \vert \vert {p}_{θ} ( Z \vert y , \overline{x} ) )$$

$$= {𝔼}_{z ∼ {q}_{ϕ} ( \cdot \vert y , \overline{x} )} [ - ln \frac{p ( z ) {p}_{θ} ( y , \tilde{x} \vert z , m )}{{q}_{ϕ} ( z \vert y , \overline{x} )} ]$$

$$= : - {ℒ}_{JELBO} ( θ , ϕ )$$

where D_{KL}(q(Z)∥p(Z)):=∫dzq(z)ln[q(z)/p(z)] is the Kullback-Leibler divergence and q_{ϕ}(z|y,x̅):=q(z|ϕ(y,x̅)) is a conditional density function defined by ϕ. [symbol] _{JELBO}(θ, φ) is referred to as the joint evidence lower bound (JELBO). Since JELBO is an expectation of a tractable function, the exemplary methods approximate JELBO with Monte-Carlo sampling.

JELBO is thus defined with two probability networks, a decoder θ, which defines joint evidence ln p_{θ}(\tilde{x},y|m) and an encoder ϕ, which lower-bounds the evidence via KL divergence. The expectation is unbiasedly estimated with Monte-Carlo sampling.

Regarding the Marginal Evidence Upper Bound (MEUBO), to bound the second term, the exemplary methods start with applying the χ-evidence upper bound (CUBO). For any real numbers α>1, CUBO is derived as follows:

$$- ln {p}_{θ} ( \tilde{x} \vert m )$$

$$\leq - ln {p}_{θ} ( \tilde{x} \vert m ) + ( 1 - {α}^{- 1} ) {D}_{α} ( {p}_{θ} ( Z \vert \overline{x} ) \vert \vert {q}_{ψ} ( Z \vert \overline{x} ) )$$

$$= \frac{1}{α} {𝔼}_{z ∼ {q}_{ψ} ( \cdot \vert \overline{x} )} [ ln \frac{p ( z ) {p}_{θ} ( \tilde{x} \vert z , m )}{{q}_{ψ} ( z \vert \overline{x} )} ]$$

$$= : {ℒ}_{CUBO} ( θ , ψ ) ,$$

where

$${D}_{α} ( p ( Z ) \vert \vert q ( Z ) ) := \frac{1}{α - 1} ln \int dz {p}^{α} ( Z ) {q}^{1 - α} ( z )$$

denotes the α-Renyi divergence and q_{ψ}(z|x̅):=q(z|ψ(x̅)) is a conditional density function defined by ψ. The exemplary methods consider the case where α=2 in particular, but the following method can be equally applicable to the other cases.

Note here, unlike JELBO, CUBO is not unbiasedly approximated because of the logarithm wrapping the expectation. To work around this issue, the exemplary embodiments introduce an additional divergence measure referred to as the exponential divergence measure Ψₐ(t):=(e^{at}−αt−1)/α, t∈ [symbol] , along with a surrogate variational network ξ: [symbol] ^{d}→ [symbol] .

It is observed that Ψα(t)≥0 for all t∈ [symbol] and thus:

$${ℒ}_{CUBO} ( θ , ψ ) \leq {ℒ}_{CUBO} ( θ , ψ ) + {Ψ}_{α} ( {ℒ}_{CUBO} ( θ , ψ ) - ξ ( \overline{x} ) ) = ( \frac{{e}^{- α ξ ( \overline{x} )}}{α} ) {{𝔼}_{z ∼ {q}_{ψ ( \cdot \vert \overline{χ} )}} [ \frac{p ( z ) {p}_{θ} ( \tilde{x} \vert z , m )}{{q}_{ψ} ( z \vert \overline{x} )} ]}^{α} + ξ ( \overline{x} ) - \frac{1}{α} = \text{:} {ℒ}_{MEUBO} ( θ , ψ , ξ ) ,$$

where the right-hand side is referred to as the marginal evidence upper bound (MEUBO). Note that MEUBO can be unbiasedly approximated with Monte-Carlo estimation and the inequality is tight, if and only if, ξ(x̅)= [symbol] _{CUBO}(θ,ψ).

Thus, CUBO is defined with 2 probability networks, that is, decoder θ, which defines the marginal evidence ln p_{θ}(x̅) and encoder ψ, which upper-bounds the evidence via α-Renyi divergence (α>1). However, the expectation cannot be unbiasedly estimated with Monte-Carlo sampling because of the logarithm wrapping the expectation. To solve such issue, the exemplary embodiments construct MEUBO, an upper bound on CUBO, with an exponential divergence measure and a surrogate network ξ:x̅ [symbol] ξ(x̅)∈ [symbol] . Unbiased Monte-Carlo estimation is now possible.

Regarding Discriminative Variational Autoencoders (DVAE), combining JELBO and MEUBO, an upper bound of the objective function can be had:

[symbol]

_{DVAE}(θ,ϕ,ψ,ξ):= [symbol] _{MEUBO}(θ,ψ,ξ)− [symbol] _{JELBO}(θ,ϕ)≥ [symbol] (θ).

The objective gap is tight if the variational networks are expressive enough, e.g.,

q_{ϕ}(z|y,x̅)≈p_{θ}(z|y,x̅),q_{ψ}(z|x̅)≈p_{θ}(z|x̅) and ξ(x̅)≈ln p(\tilde{x}|m).

Since both JELBO and MEUBO can be unbiasedly approximated, the exemplary method can employ stochastic gradient-based optimization methods to minimize [symbol] _{DVAE}. Specifically, JELBO for the i-th instance is approximated with

$${\hat{L}}_{JELBO}^{i} ( θ , ϕ ) := \frac{1}{{k}_{ϕ}} {\sum }_{z ∈ {S}_{ϕ}} ln \frac{p ( z ) {p}_{θ} ( {y}^{i} , {\tilde{x}}^{i} \vert z , m )}{{q}_{ϕ} ( z \vert {y}^{i} , {\overline{x}}^{i} )}$$

and MEUBO for the i-th instance is approximated with:

$${\hat{L}}_{MEUBO}^{i} ( θ , ψ , ξ ) := \frac{{e}^{- α ξ ( {\overline{x}}^{i} )}}{α {k}_{ψ}} {\sum }_{z ∈ {S}_{ψ}} {[ \frac{p ( z ) {p}_{θ} ( {\tilde{x}}^{i} \vert z , m )}{{q}_{ψ} ( z \vert {\overline{x}}^{i} )} ]}^{α} + ξ ( {\overline{x}}^{i} ) - \frac{1}{α}$$

where Sϕ and Sψ are Monte-Carlo samples drawn from q_{ϕ}(z|yⁱ,x̅ⁱ) and q_{ψ}(z|x̅ⁱ) with |S_{ϕ}|=k_{ϕ} and |S_{ψ}|=k_{ψ}, respectively. The gradients of these functions are taken with any standard automatic differentiation libraries, using, e.g., the reparametrization trick or the REINFORCE trick.

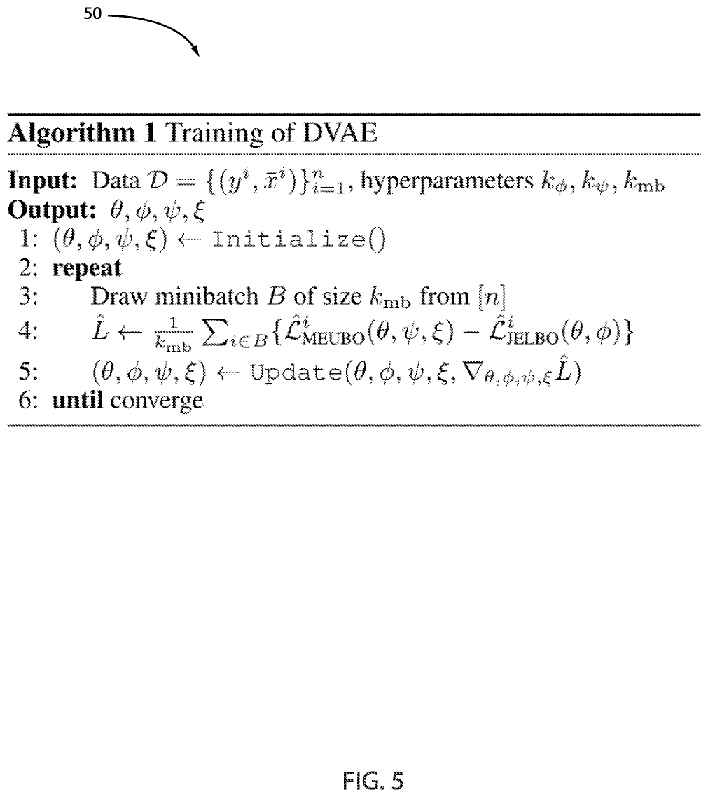

Finally, since the actual objective function is the summation of individual losses [symbol] _{DVAE}ⁱ:= [symbol] _{MEUBO}ⁱ− [symbol] _{JELBO}ⁱ over all the patients, the exemplary embodiments can draw a minibatch of patients of size k_{mb} for each iteration. The exemplary methods refer to the resulting inference method as the discriminative variational autoencoder (DVAE), which is identified with the quadruple of neural networks (θ, ϕ, ψ, ξ), a depicted in Algorithm 1 (FIG. 5).

Regarding the Importance-Weighted MEUBO, the exemplary methods also consider an improvement over the MEUBO estimate to reduce the variance.

The new estimate is given by introducing the importance sampling with respect to the midpoint distribution q̅_{ψ}(z|x̅):=(p(z)+q_{ψ}(z|x̅))/2,

$${\hat{L}}_{I W - M E U B O} ( θ , ψ , ξ ) := \frac{{e}^{- α ξ ( \overline{x} )}}{α {k}_{ψ}} {\sum }_{z ∈ {S}_{ψ}} \frac{{p}^{α} ( z ) {p}_{θ}^{α} ( \tilde{x} \vert z , m )}{{q}_{ψ}^{α - 1} ( z \vert \overline{x} ) {\overline{q}}_{ψ} ( z \vert \overline{x} )} + ξ ( \overline{x} ) - \frac{1}{α}$$

where S_{ψ} is drawn from q̅_{ψ}(z|x̅) Note that the exemplary methods can still use the reparametrization trick with [symbol] _{IW-MEUBO} since the proposal distribution is a mixture of a constant distribution p(z) and a reparametrizable distribution q_{ψ}(z|x̅). Moreover, the importance-weighted estimate behaves better than the original one in terms of their variances:

Regarding a first theorem:

Let V:=Var[ [symbol] _{MEUBO}(θ,ψ,ξ)] and V_{IW}:=Var[ [symbol] _{IW-MEUBO}(θ,ψ,ξ)] denote the variances of the estimates, respectively.

Also let Δ:= [symbol] _{DVAE}(θ,ϕ,ψ,ξ)− [symbol] (θ) denote the objective gap. Then

$${v}_{I W} \leq ( 1 ⩓ \frac{β}{{( {k}_{ψ} v )}^{\frac{1}{2 α}}} ) [ 2 v + \frac{{e}^{\sqrt{8 α Δ}}}{{k}_{ψ}} ] ,$$

where Λ denotes the minimum operator and β:=2e^{-ξ(x̅)}sup_{z}p_{θ}(\tilde{x}|z,m).

In other words, the variance of [symbol] _{IW-MEUBO} is asymptotically smaller than [symbol] _{MEUBO} by an exponent of

$$1 - \frac{1}{2 α} ,$$

while, in a non-asymptotic sense, it is still favorable up to an additive and a multiplicative constants if the objective gap Δ is bounded. In particular, [symbol] _{IW-MEUBO} is stabler than [symbol] _{MEUBO} in bad conditions, e.g., where q is largely misspecified, owing to the exponent

$$1 - \frac{1}{2 α} < 1 .$$

Regarding the proof, let the summand of MEUBO be denoted as:

$${w}^{α} ( z ) : = {[ \frac{p ( z ) {p}_{θ} ( \tilde{x} \vert z , m )}{{q}_{ψ} ( z \vert \overline{x} )} ]}^{α}$$

and the importance weight as:

$$γ ( z ) : = \frac{{q}_{ψ} ( z \vert \overline{x} )}{{\overline{q}}_{ψ} ( z \vert \overline{x} )} .$$

Then,

$$v = A ( {𝔼}_{z ∼ ψ} [ {w}^{2 α} ( z ) ] - C ) , \\ {v}_{IW} = A ( {𝔼}_{z ∼ ψ} [ γ ( z ) {w}^{2 α} ( z ) ] - C ) ,$$

where

$$A := \frac{{e}^{- 2 α ξ ( \overline{x} )}}{{α}^{2} {k}_{ψ}} ,$$

C:=e^{2α} [symbol] ^{_{CUBO}}^{(θ,ψ)} and [symbol] _{z˜ψ} is a shorthand for the expectation with respect to z˜q_{ψ}(z|x̅).

Now it is observed that γ(z)≤2 and thus:

$${v}_{IW} \leq A ( 2 ( \frac{v}{A} + C ) - C ) = 2 v + AC .$$

Moreover, the exemplary methods also have γ(z)≤2Mw⁻¹(z) for

M:=sup_{z}p(x̅|z,m).

And thus

$${𝒱}_{I W} \leq A ( 2 M {𝔼}_{z ∼ ψ} [ {w}^{2 α - 1} ( z ) ] - C )$$

$$\leq A ( 2 {M ( \frac{𝒱}{A} + C )}^{1 - \frac{1}{2 α}} - C ) ( Jensen ' s inequality )$$

$$= 2 M {{A}^{\frac{1}{2 α}} ( 𝒱 + A C )}^{1 - \frac{1}{2 α}} - A C$$

$$\leq 2 {M ( \frac{A}{𝒱} )}^{\frac{1}{2 α}} ( 2 𝒱 + A C ) . ( 𝒱 , A , C \geq 0 )$$

Finally, the conclusion follows with the simplification on the right-hand sides,

$$A \leq \frac{{e}^{- 2 α ξ ( \overline{x} )}}{{k}_{ψ}} , AC \leq \frac{1}{{k}_{ψ}} {e}^{2 α ( {ℒ}_{CUBO} ( θ , ψ ) - ξ ( \overline{x} ) )}$$

$$and$$

$${ℒ}_{CUBO} ( θ , ψ ) - ξ ( \overline{x} ) \leq \sqrt{\frac{2}{α} {Ψ}_{α} ( {ℒ}_{CUBO} ( θ , ψ ) - ξ ( \overline{x} ) )} \leq \sqrt{\frac{2}{α} Δ} .$$

The prediction procedure can be as follows:

Consider using an already-trained DVAE (θ, ϕ, ψ, ξ) to make a prediction on unseen patients given their incomplete covariates x̅=x̅ⁿ⁺¹.

Since the conditional density p_{θ}(y|x̅) can be intractable in general, the exemplary methods approximate it with Monte-Carlo sampling and the variational distribution q_{ψ}(z|x̅) instead. Namely, the approximated conditional distribution is given by:

$${\hat{p}}_{θ} ( y \vert \overline{x} ) : = \frac{1}{{k}_{pred}} {\sum }_{s = 1}^{{k}_{pred}} δ ( y - {\hat{y}}^{s} ) , {k}_{pred} \geq 1 ,$$

Where ŷ^{s}˜p_{θ}(y|\hat{z}^{s}) and \hat{z}^{s}˜q_{ψ}(z|x̅), s∈[k_{pred}], are independently drawn Monte-Carlo samples. This procedure is justified as follows.

Regarding the second theorem, let p̅_{θ}(y|x̅):= [symbol] [\hat{p}_{θ}(y|x̅)] be the mean of the approximation with respect to the Monte-Carlo samples. Then, the approximation error with respect to the KL divergence is bounded with the objective gap,

$${D}_{KL} ( {p}_{θ} ( Y \vert \overline{x} ) \vert \vert {\overline{p}}_{θ} ( Y \vert \overline{x} ) ) \leq \frac{α}{α - 1} Δ ,$$

where Δ is defined in the first theorem.

In other words, if the variational networks are trained enough that the objective gap is small, so is the approximation error of p̅_{θ}, which is the weak large sample limit (k_{pred}→∞) of the actual predictor \hat{p}_{θ}.

The proof is provided as follows:

According to the information processing inequality, the exemplary methods have D_{KL}(p_{θ}(Y|x̅)∥p̅_{θ}(Y|x̅)≤D_{KL}(p_{θ}(Z|x̅)∥q_{ψ}(Z|x̅)).

Moreover, by the construction of [symbol] _{MEUBO}, the exemplary methods have (1−α⁻¹)D_{α}(p_{θ}(Z|x̅)∥q_{ψ}(Z|x̅))≤Δ.

The desired result is seen by combining these two inequalities with the fact that D_{KL}(p∥q)≤D_{α}(p∥q) for all α>1.

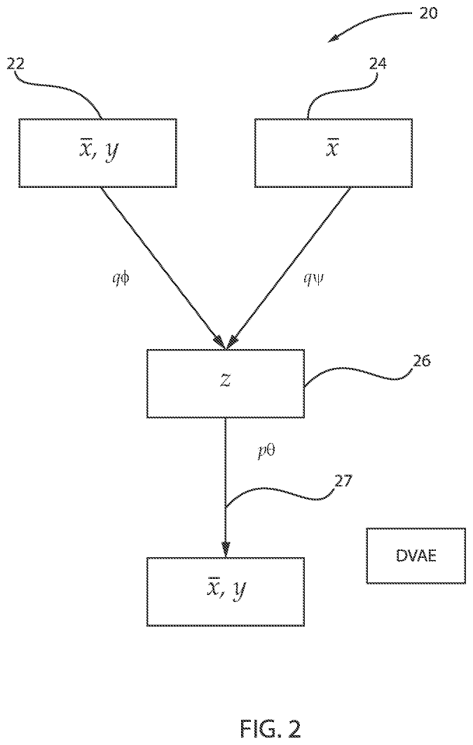

FIG. 2 is an exemplary computation graph 20 of the DVAE 10 of FIG. 1, in accordance with an embodiment of the present invention.

Referring back to FIG. 1, solid lines 12 denote the generative model p(m)p(z) p_{θ}(y,x|z, m) p(\tilde{x}|x, m), dashed lines 14 denote the variational approximation q_{ϕ}(z|y,x̅) to the intractable posterior given joint observations p_{θ}(z|y,x̅), chain lines 16 denote the variational approximation q_{ψ}(z|x̅) to the intractable posterior given marginal observations p_{θ}(z|x̅) with the help of the surrogate variational parameter ξ. The variational parameters (φ, ψ, ξ) are learned jointly with the generative model parameter θ. In FIG. 1, 11 denotes z (noise variable), 13 denotes x (complete covariates), 15 denotes y (outcome), 17 denotes \tilde{x} (incomplete covariates), and 19 denotes m (mask vector).

With reference to FIG. 2, three probability models (θ, ϕ, ψ), ti) are combined to form a new architecture referred to as a discriminative variational autoencoder (DVAE). In other words, the decoder p_{θ}(x̅,y|z), the joint encoder p_{ϕ}(z|x̅,y), and the marginal encoder p_{ψ}(z|x̅) are combined. JELBO is computed with the decoder and the joint encoder, whereas MEUBO is computed with the decoder and the marginal encoder. In other words, the variational approximation q_{ϕ} (22) and the variational approximation q_{ψ} (24) are provided to z (26) to output the model p_{θ} (27).

Thus, a method of computing the objective of DIGM for an incomplete covariate is provided with difference of marginal evidence upper bound ( [symbol] _{MEUBO}) and joint evidence lower bound ( [symbol] _{JELBO}) and ln p_{θ}(x̅)≤ [symbol] _{MEUBO} such that ln p_{θ}(x̅,y)≥ [symbol] _{JELBO}, such that [symbol] :=−ln p_{θ}(y|x̅)=ln p_{θ}(x̅)−ln p_{θ}(x̅,y)≤ [symbol] _{MEUBO}− [symbol] _{JELBO}.

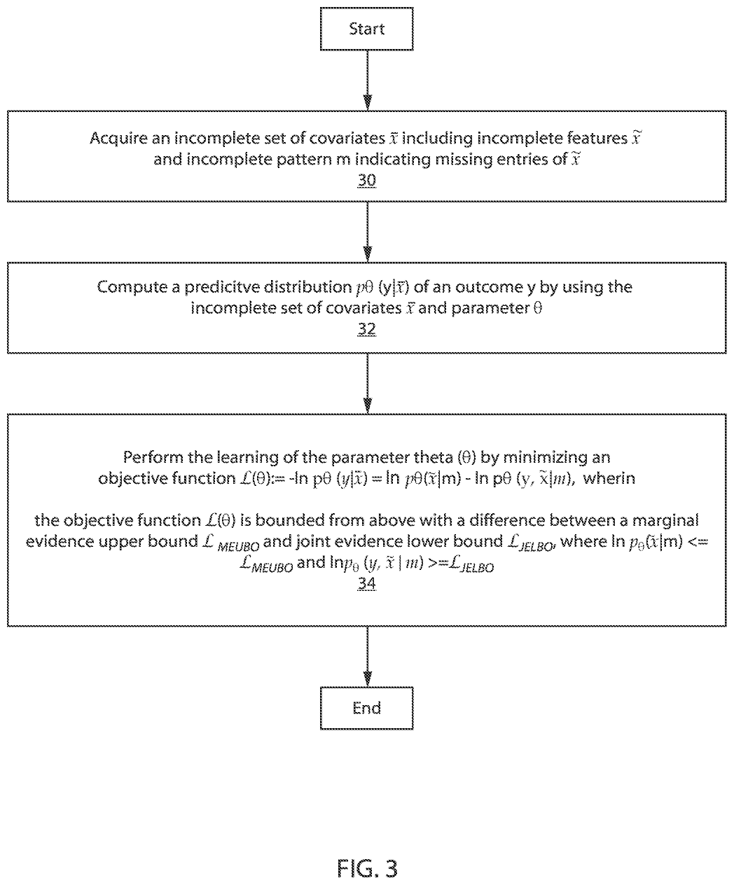

FIG. 3 is a block/flow diagram of an exemplary method for computing an objective function of discriminative inference with generative models with incomplete data, in accordance with an embodiment of the present invention.

At block 30, acquire an incomplete set of covariates x̅ including incomplete features \tilde{x} and incomplete pattern m indicating missing entries of \tilde{x}.

At block 32, compute a predictive distribution p_{θ}(y|x̅) of an outcome y by using the incomplete set of covariates x̅ and parameter θ learned.

At block 34, perform the learning of the parameter θ by minimizing an objective function [symbol] (θ):=−ln p_{θ}(y|x̅)=ln p_{θ}(\tilde{x}|m)−ln p_{θ}(y,x̅|m), wherein the objective function [symbol] (θ) is bounded from above with a difference between a marginal evidence upper bound [symbol] _{MEUBO} and joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

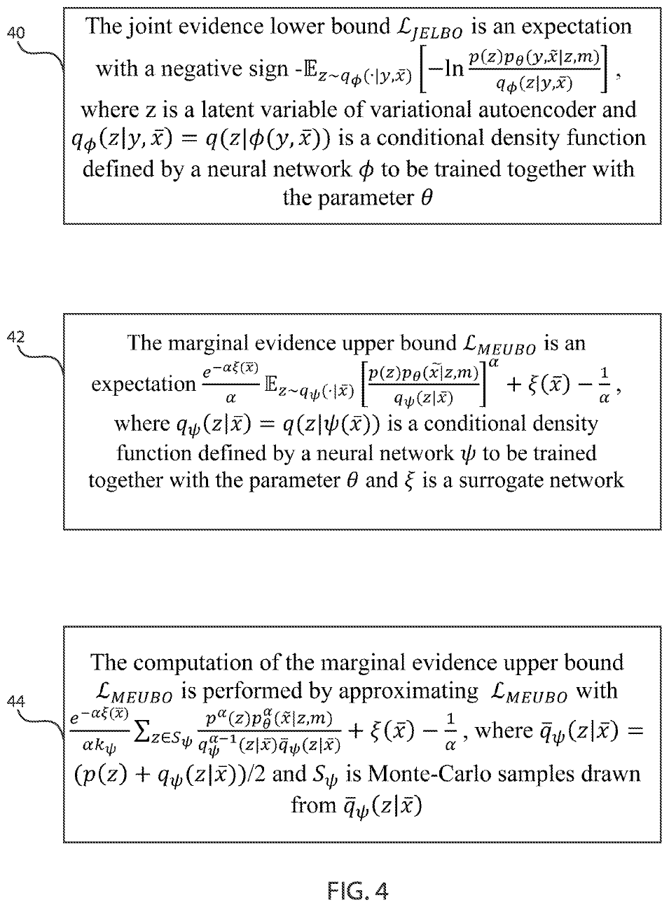

FIG. 4 illustrates joint evidence lower bound and marginal evidence upper bound equations, in accordance with an embodiment of the present invention.

At block 40, the joint evidence lower bound [symbol] _{JELBO} is an expectation with a negative

$$sign - {𝔼}_{z ∼ {q}_{ϕ} ( \cdot \vert y , \overline{x} )} [ - ln \frac{p ( z ) {p}_{θ} ( y , \tilde{x} \vert z , m )}{{q}_{ϕ} ( z \vert y , \overline{x} )} ] ,$$

where z is a latent variable of variational autoencoder and q_{ϕ}(z|y,x̅)=q(z|ϕ(y,x̅)) is a conditional density function defined by a neural network ϕ to be trained together with the parameter θ.

At block 42, the marginal evidence upper bound [symbol] _{MEUBO} is an expectation

$$\frac{{e}^{- α ξ ( \overline{x} )}}{α} {{𝔼}_{z ∼ {q}_{ψ} ( \cdot \vert \overline{x} )} [ \frac{p ( z ) {p}_{θ} ( \tilde{x} \vert z , m )}{{q}_{ψ} ( z \vert \overline{x} )} ]}^{α} + ξ ( \overline{x} ) - \frac{1}{α} ,$$

where q_{ψ}(z|x̅)=q(z|ψ(x̅)) is a conditional density function defined by a neural network to be trained together with the parameter θ and ξ is a surrogate network.

At block 44, the computation of the marginal evidence upper bound [symbol] _{MEUBO} is performed by approximating [symbol] _{MEUBO}

$$with \frac{{e}^{- α ξ ( \overline{x} )}}{α {k}_{ψ}} {\sum }_{z ∈ {S}_{ψ}} \frac{{p}^{α} ( z ) {p}_{θ}^{α} ( \tilde{x} \vert z , m )}{{q}_{ψ}^{α - 1} ( z \vert \overline{x} ) {\overline{q}}_{ψ} ( z \vert \overline{x} )} + ξ ( \overline{x} ) - \frac{1}{α} ,$$

where q̅_{ψ}(z|x̅)=(p(z)+q_{ψ}(z|x̅))/2 and S_{ψ} is Monte-Carlo samples drawn from q̅_{ψ}(z|x̅).

FIG. 5 is an exemplary algorithm 50 for training the DVAE, in accordance with an embodiment of the present invention.

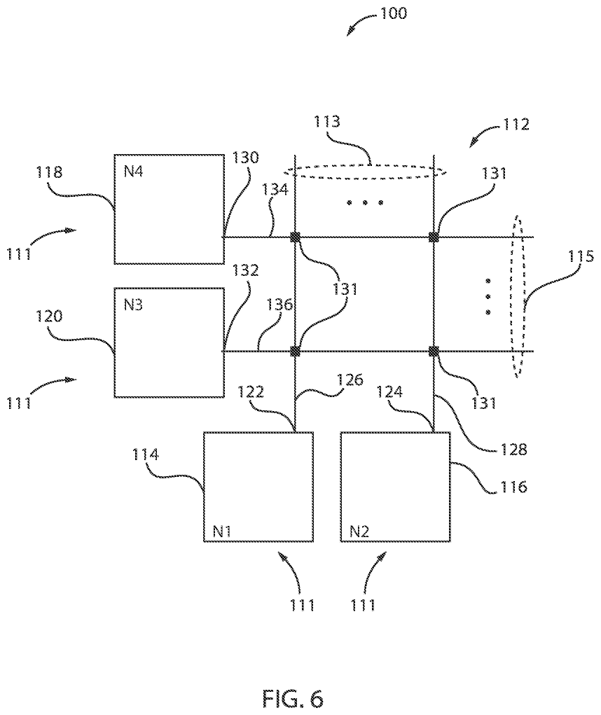

FIG. 6 is an exemplary neuromorphic and synaptronic network including a crossbar of electronic synapses interconnecting electronic neurons and axons, in accordance with an embodiment of the present invention. Such ANNs can incorporate the DVAE.

The example tile circuit 100 has a crossbar 112 in accordance with an embodiment of the invention. In one example, the overall circuit can include an “ultra-dense crossbar array” that can have a pitch in the range of about 10 nm to 500 nm. However, one skilled in the art can contemplate smaller and larger pitches as well. The neuromorphic and synaptronic circuit 100 includes the crossbar 112 interconnecting a plurality of digital neurons 111 including neurons 114, 116, 118 and 120. These neurons 111 are also referred to herein as “electronic neurons.” For illustration purposes, the example circuit 100 provides symmetric connections between the two pairs of neurons (e.g., N1 and N3). However, embodiments of the invention are not only useful with such symmetric connection of neurons, but also useful with asymmetric connection of neurons (neurons N1 and N3 need not be connected with the same connection). The cross-bar in a tile accommodates the appropriate ratio of synapses to neurons, and, hence, need not be square.

In the example circuit 100, the neurons 111 are connected to the crossbar 112 via dendrite paths/wires (dendrites) 113 such as dendrites 126 and 128. Neurons 111 are also connected to the crossbar 112 via axon paths/wires (axons) 115 such as axons 134 and 136. Neurons 114 and 116 are dendritic neurons and neurons 118 and 120 are axonal neurons connected with axons 113. Specifically, neurons 114 and 116 are shown with outputs 122 and 124 connected to dendrites (e.g., bitlines) 126 and 128, respectively. Axonal neurons 118 and 120 are shown with outputs 130 and 132 connected to axons (e.g., wordlines or access lines) 134 and 136, respectively.

When any of the neurons 114, 116, 118 and 120 fire, they will send a pulse out to their axonal and to their dendritic connections. Each synapse provides contact between an axon of a neuron and a dendrite on another neuron and with respect to the synapse, the two neurons are respectively called pre-synaptic and post-synaptic.

Each connection between dendrites 126, 128 and axons 134, 136 are made through a digital synapse device 131 (synapse). The junctions where the synapse devices are located can be referred to herein as “cross-point junctions.” In general, in accordance with an embodiment of the invention, neurons 114 and 116 will “fire” (transmit a pulse) in response to the inputs they receive from axonal input connections (not shown) exceeding a threshold.

Neurons 118 and 120 will “fire” (transmit a pulse) in response to the inputs they receive from external input connections (not shown), usually from other neurons, exceeding a threshold. In one embodiment, when neurons 114 and 116 fire, they maintain a postsynaptic spike-timing-dependent plasticity (STDP) (post-STDP) variable that decays. For example, in one embodiment, the decay period can be 50 μs (which is 1000× shorter than that of actual biological systems, corresponding to 1000× higher operation speed). The post-STDP variable is employed to achieve STDP by encoding the time since the last firing of the associated neuron. Such STDP is used to control long-term potentiation or “potentiation,” which in this context is defined as increasing synaptic conductance. When neurons 118, 120 fire they maintain a pre-STDP (presynaptic-STDP) variable that decays in a similar fashion as that of neurons 114 and 116.

An external two-way communication environment can supply sensory inputs and consume motor outputs. Digital neurons 111 implemented using complementary metal oxide semiconductor (CMOS) logic gates receive spike inputs and integrate them. In one embodiment, the neurons 111 include comparator circuits that generate spikes when the integrated input exceeds a threshold. In one embodiment, synapses are implemented using flash memory cells, wherein each neuron 111 can be an excitatory or inhibitory neuron (or both). Each learning rule on each neuron axon and dendrite are reconfigurable as described below. This assumes a transposable access to the crossbar memory array. Neurons that spike are selected one at a time sending spike events to corresponding axons, where axons could reside on the core or somewhere else in a larger system with many cores.

The term electronic neuron as used herein represents an architecture configured to simulate a biological neuron. An electronic neuron creates connections between processing elements that are roughly functionally equivalent to neurons of a biological brain. As such, a neuromorphic and synaptronic system including electronic neurons according to embodiments of the invention can include various electronic circuits that are modeled on biological neurons, though they can operate on a faster time scale (e.g., 1000×) than their biological counterparts in many useful embodiments. Further, a neuromorphic and synaptronic system including electronic neurons according to embodiments of the invention can include various processing elements (including computer simulations) that are modeled on biological neurons. Although certain illustrative embodiments of the invention are described herein using electronic neurons including electronic circuits, the present invention is not limited to electronic circuits. A neuromorphic and synaptronic system according to embodiments of the invention can be implemented as a neuromorphic and synaptronic architecture including circuitry, and additionally as a computer simulation.

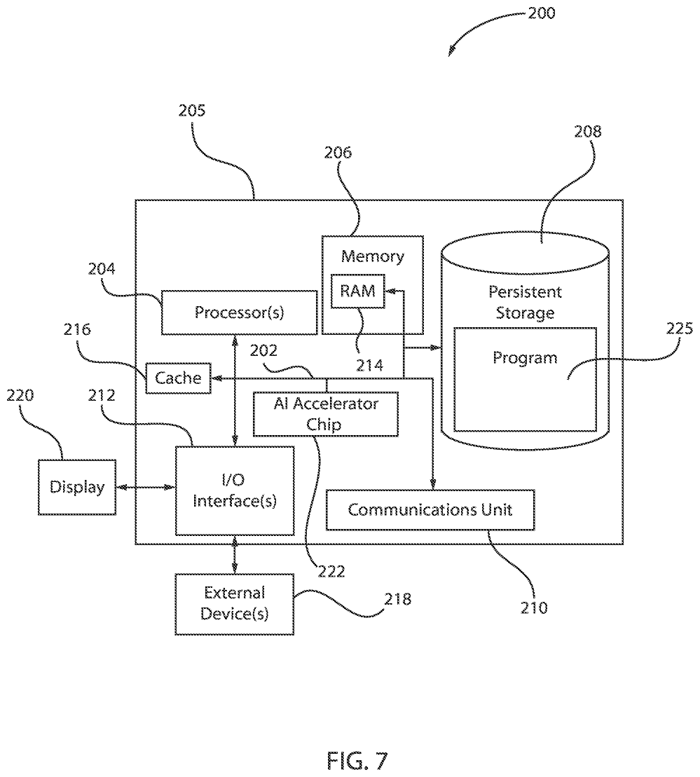

FIG. 7 is a block diagram of components of a computing system including a computing device employing the algorithm of FIG. 5 for training the DVAE via an artificial intelligence (AI) accelerator chip, in accordance with an embodiment of the present invention.

FIG. 7 depicts a block diagram of components of system 200, which includes computing device 205. It should be appreciated that FIG. 7 provides only an illustration of one implementation and does not imply any limitations with regard to the environments in which different embodiments can be implemented. Many modifications to the depicted environment can be made.

Computing device 205 includes communications fabric 202, which provides communications between computer processor(s) 204, memory 206, persistent storage 208, communications unit 210, and input/output (I/O) interface(s) 212. Communications fabric 202 can be implemented with any architecture designed for passing data and/or control information between processors (such as microprocessors, communications and network processors, etc.), system memory, peripheral devices, and any other hardware components within a system. For example, communications fabric 202 can be implemented with one or more buses.

Memory 206, cache memory 216, and persistent storage 208 are computer readable storage media. In this embodiment, memory 206 includes random access memory (RAM) 214. In another embodiment, the memory 206 can be flash memory. In general, memory 206 can include any suitable volatile or non-volatile computer readable storage media.

In some embodiments of the present invention, deep learning program 225 is included and operated by AI accelerator chip 222 as a component of computing device 205. In other embodiments, deep learning program 225 is stored in persistent storage 208 for execution by AI accelerator chip 222 in conjunction with one or more of the respective computer processors 204 via one or more memories of memory 206. In this embodiment, persistent storage 208 includes a magnetic hard disk drive. Alternatively, or in addition to a magnetic hard disk drive, persistent storage 208 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 208 can also be removable. For example, a removable hard drive can be used for persistent storage 208. Other examples include optical and magnetic disks, thumb drives, and smart cards that are inserted into a drive for transfer onto another computer readable storage medium that is also part of persistent storage 208.

Communications unit 210, in these examples, provides for communications with other data processing systems or devices, including resources of distributed data processing environment. In these examples, communications unit 210 includes one or more network interface cards. Communications unit 210 can provide communications through the use of either or both physical and wireless communications links. Deep learning program 225 can be downloaded to persistent storage 208 through communications unit 210.

I/O interface(s) 212 allows for input and output of data with other devices that can be connected to computing system 200. For example, I/O interface 212 can provide a connection to external devices 218 such as a keyboard, keypad, a touch screen, and/or some other suitable input device. External devices 218 can also include portable computer readable storage media such as, for example, thumb drives, portable optical or magnetic disks, and memory cards.

Display 220 provides a mechanism to display data to a user and can be, for example, a computer monitor.

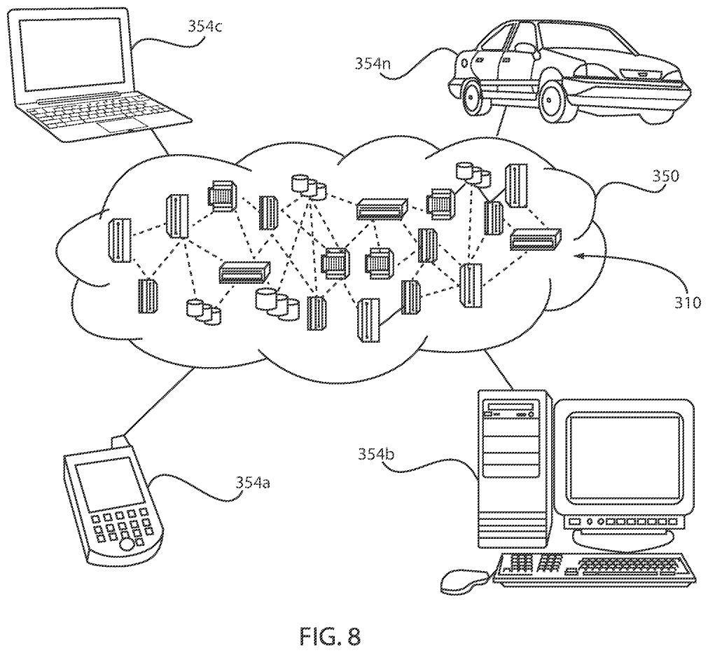

FIG. 8 is a block/flow diagram of an exemplary cloud computing environment, in accordance with an embodiment of the present invention.

It is to be understood that although this invention includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 8, illustrative cloud computing environment 350 is depicted for enabling use cases of the present invention. As shown, cloud computing environment 350 includes one or more cloud computing nodes 310 with which local computing devices used by cloud consumers, such as, for example, personal digital assistant (PDA) or cellular telephone 354A, desktop computer 354B, laptop computer 354C, and/or automobile computer system 354N can communicate. Nodes 310 can communicate with one another. They can be grouped (not shown) physically or virtually, in one or more networks, such as Private, Community, Public, or Hybrid clouds as described hereinabove, or a combination thereof. This allows cloud computing environment 350 to offer infrastructure, platforms and/or software as services for which a cloud consumer does not need to maintain resources on a local computing device. It is understood that the types of computing devices 354A-N shown in FIG. 8 are intended to be illustrative only and that computing nodes 310 and cloud computing environment 350 can communicate with any type of computerized device over any type of network and/or network addressable connection (e.g., using a web browser).

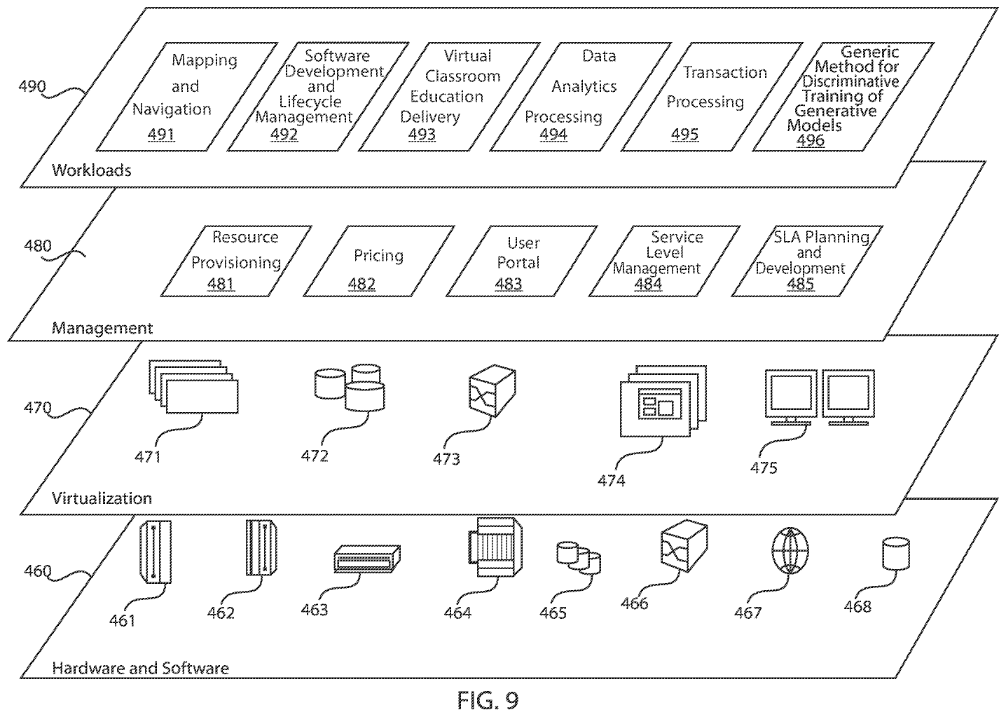

FIG. 9 is a schematic diagram of exemplary abstraction model layers, in accordance with an embodiment of the present invention. It should be understood in advance that the components, layers, and functions shown in FIG. 9 are intended to be illustrative only and embodiments of the invention are not limited thereto. As depicted, the following layers and corresponding functions are provided:

Hardware and software layer 460 includes hardware and software components. Examples of hardware components include: mainframes 461; RISC (Reduced Instruction Set Computer) architecture based servers 462; servers 463; blade servers 464; storage devices 465; and networks and networking components 466. In some embodiments, software components include network application server software 467 and database software 468.

Virtualization layer 470 provides an abstraction layer from which the following examples of virtual entities can be provided: virtual servers 471; virtual storage 472; virtual networks 473, including virtual private networks; virtual applications and operating systems 474; and virtual clients 475.

In one example, management layer 480 can provide the functions described below. Resource provisioning 481 provides dynamic procurement of computing resources and other resources that are utilized to perform tasks within the cloud computing environment. Metering and Pricing 482 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 483 provides access to the cloud computing environment for consumers and system administrators. Service level management 484 provides cloud computing resource allocation and management such that required service levels are met. Service Level Agreement (SLA) planning and fulfillment 485 provide pre-arrangement for, and procurement of, cloud computing resources for which a future requirement is anticipated in accordance with an SLA.

Workloads layer 490 provides examples of functionality for which the cloud computing environment can be utilized. Examples of workloads and functions which can be provided from this layer include: mapping and navigation 441; software development and lifecycle management 492; virtual classroom education delivery 493; data analytics processing 494; transaction processing 495; and generic method for discriminative training of generative models 496.

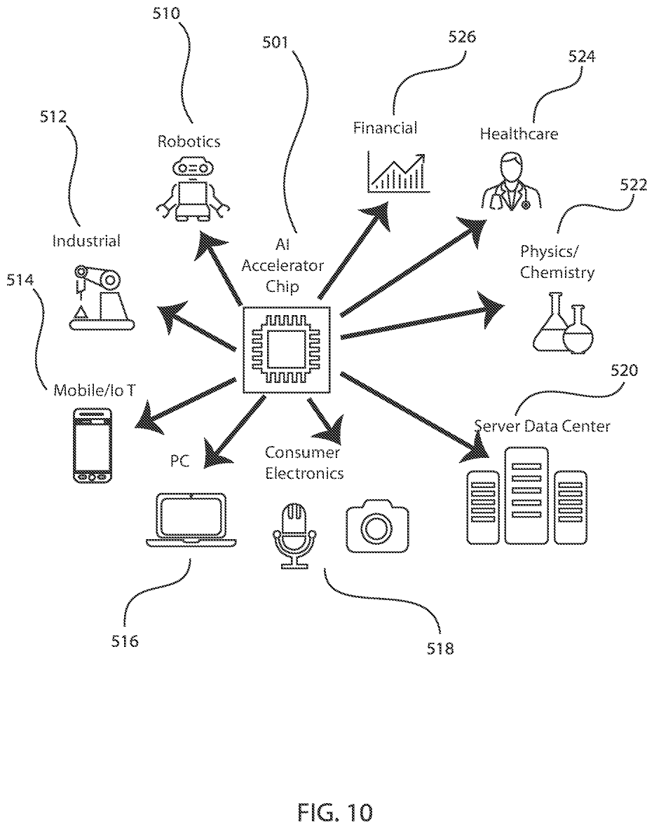

FIG. 10 illustrates practical applications for employing the DVAE via an AI accelerator chip, in accordance with an embodiment of the present invention.

The artificial intelligence (AI) accelerator chip 501 can be used in a wide variety of practical applications, including, but not limited to, robotics 510, industrial applications 512, mobile or Internet-of-Things (IoT) 514, personal computing 516, consumer electronics 518, server data centers 520, physics and chemistry applications 522, healthcare applications 524, and financial applications 526.

For example, Robotic Process Automation or RPA 510 enables organizations to automate tasks, streamline processes, increase employee productivity, and ultimately deliver satisfying customer experiences. Through the use of RPA 510, a robot can perform high volume repetitive tasks, freeing the company's resources to work on higher value activities. An RPA Robot 510 emulates a person executing manual repetitive tasks, making decisions based on a defined set of rules, and integrating with existing applications. All of this while maintaining compliance, reducing errors, and improving customer experience and employee engagement.

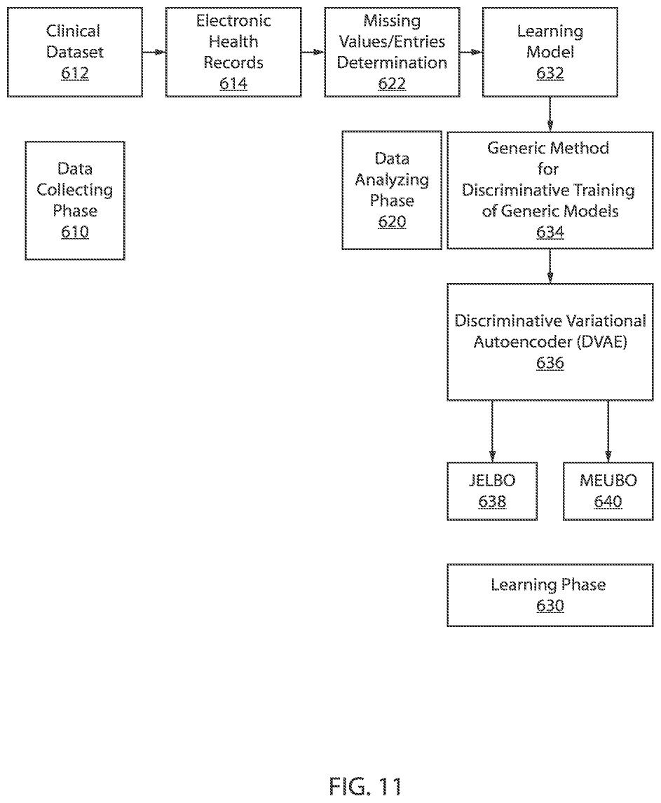

FIG. 11 is a block/flow diagram of a practical application including health care records for employing the DVAE via the AI accelerator chip, in accordance with an embodiment of the present invention.

In a data collecting phase 610, a clinical dataset 612 includes electronic health records (EHR) 614. In a data analyzing phase 620, the missing values or entries are determined at block 622. In a learning phase 630, a learning model 632 is employed, the learning model 632 using a generic method for discriminative training of generic models in block 634 that utilizes a discriminative variational autoencoder (DVAE) 636. The DVAE 636 computes JELBO 638 and MEUBO 640.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory, a read-only memory, an erasable programmable read-only memory (EPROM or Flash memory), a static random access memory, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to at least one processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules. These computer readable program instructions can also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks or modules.

The computer readable program instructions can also be loaded onto a computer, other programmable data processing apparatus, or other device to cause a series of operational blocks/steps to be performed on the computer, other programmable apparatus or other device to produce a computer implemented process, such that the instructions which execute on the computer, other programmable apparatus, or other device implement the functions/acts specified in the flowchart and/or block diagram block or blocks or modules.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This can be extended, as readily apparent by one of ordinary skill in this and related arts, for as many items listed.

Having described preferred embodiments of a method for computing an objective function of discriminative inference with generative models with incomplete data in which some of entries are missing (which are intended to be illustrative and not limiting), it is noted that modifications and variations can be made by persons skilled in the art in light of the above teachings. It is therefore to be understood that changes may be made in the particular embodiments described which are within the scope of the invention as outlined by the appended claims. Having thus described aspects of the invention, with the details and particularity required by the patent laws, what is claimed and desired protected by Letters Patent is set forth in the appended claims.

1. A computer-implemented method for computing an objective function of discriminative inference with generative models with incomplete data in which some of entries are missing, comprising:
acquiring an incomplete set of covariates x̅ including incomplete features \tilde{x} and an incomplete pattern m indicating missing entries of the incomplete features \tilde{x}; and
computing a predictive distribution p_{θ}(y|x̅) of an outcome y by using the incomplete set of covariates x̅ and a parameter θ, the parameter θ being unknown;
wherein learning of the parameter θ is performed by minimizing an objective function [symbol] (θ):=−ln p_{θ}(y|x̅)=ln p_{θ}(\tilde{x}|m)−ln p_{θ}(y,\tilde{x}|m), and the objective function [symbol] (θ) is bounded with a difference between a marginal evidence upper bound [symbol] _{MEUBO} and a joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

2. The computer-implemented method of claim 1, wherein the joint evidence lower bound [symbol] _{JELBO} is an expectation with a negative sign
$$- {𝔼}_{z ∼ {q}_{ϕ} ( \cdot \vert y , \overline{x} )} [ - ln \frac{p ( z ) {p}_{θ} ( y , \tilde{x} \vert z , m )}{{q}_{ϕ} ( z \vert y , \overline{x} )} ] ,$$
where z is a latent variable of a variational autoencoder and q_{ϕ}(z|y,x̅)=q(z|ϕ(y,x̅)) is a conditional density function defined by a neural network ϕ to be trained together with the parameter θ.

3. The computer-implemented method of claim 2, wherein the marginal evidence upper bound [symbol] _{MEUBO} is an expectation
$$\frac{{e}^{- α ξ ( \overline{x} )}}{α} {{E}_{z ∼ {q}_{ψ} ( \cdot \vert \overline{x} )} [ \frac{p ( z ) {p}_{θ} ( \tilde{x} \vert z , m )}{q ψ ( z \vert \overline{x} )} ]}^{α} + ξ ( \overline{x} ) - \frac{1}{α} ,$$
where q_{ψ}(z|x̅)=q(z|ψ(x̅)) is a conditional density function defined by a neural network ψ to be trained together with the parameter θ and ξ is a surrogate network.

4. The computer-implemented method of claim 3, wherein computation of the marginal evidence upper bound [symbol] _{MEUBO} is performed by approximating [symbol] _{MEUBO} with
$$\frac{{e}^{- α ξ ( \overline{x} )}}{α {k}_{ψ}} {\sum }_{z ∈ {S}_{ψ}} \frac{{p}^{α} ( z ) {p}_{θ}^{α} ( \tilde{x} \vert z , m )}{{q}_{ψ}^{α - 1} ( z \vert \overline{x} ) \overline{q} ψ ( z \vert \overline{x} )} + ξ ( \overline{x} ) - \frac{1}{α} ,$$
where q̅_{ψ}(z|x̅)=(p(z)+q_{ψ}(z|x̅))/2 and S_{ψ} is Monte-Carlo samples drawn from q̅_{ψ}(z|x̅).

5. The computer-implemented method of claim 1, wherein a discriminative variational autoencoder (DVAE) performs discriminative inference with generative models (DIGM) with the incomplete set of covariates x̅.

6. The computer-implemented method of claim 5, wherein the DVAE includes a generative network, two variational networks, and a surrogate network.

7. The computer-implemented method of claim 6, wherein stochastic gradient-based optimization is employed to minimize the objective function.

8. A computer program product for computing an objective function of discriminative inference with generative models with incomplete data in which some of entries are missing, the computer program product comprising a computer readable storage medium having program instructions embodied therewith, the program instructions executable by a computer to cause the computer to:
acquire an incomplete set of covariates x̅ including incomplete features \tilde{x} and an incomplete pattern m indicating missing entries of the incomplete features \tilde{x}; and
compute a predictive distribution p_{θ}(y|x̅) of an outcome y by using the incomplete set of covariates x̅ and a parameter θ, the parameter θ being unknown;
wherein learning of the parameter θ is performed by minimizing an objective function [symbol] (θ):=−ln p_{θ}(y|x̅)=ln p_{θ}(\tilde{x}|m)−ln p_{θ}(y,\tilde{x}|m), and the objective function [symbol] (θ) is bounded with a difference between a marginal evidence upper bound [symbol] _{MEUBO} and a joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.

9. The computer program product of claim 8, wherein the joint evidence lower bound [symbol] _{JELBO} is an expectation with a negative sign
$$- {𝔼}_{z ∼ {q}_{ϕ} ( \cdot \vert y , \overline{x} )} [ - ln \frac{p ( z ) {p}_{θ} ( y , \tilde{x} \vert z , m )}{{q}_{ϕ} ( z \vert y , \overline{x} )} ] ,$$
where z is a latent variable of a variational autoencoder and q_{ϕ}(z|y,x̅)=q(z|ϕ(y,x̅)) is a conditional density function defined by a neural network ϕ to be trained together with the parameter θ.

10. The computer program product of claim 9, wherein the marginal evidence upper bound [symbol] _{MEUBO} is an expectation
$$\frac{{e}^{- α ξ ( \overline{x} )}}{α} {{𝔼}_{z ∼ {q}_{ψ} ( \cdot \vert \overline{x} )} [ \frac{p ( z ) {p}_{θ} ( \tilde{x} \vert z , m )}{q ψ ( z \vert \overline{x} )} ]}^{α} + ξ ( \overline{x} ) - \frac{1}{α} ,$$
where q_{ψ}(z|x̅)=q(z|ψ(x̅)) is a conditional density function defined by a neural network ψ to be trained together with the parameter θ and ξ is a surrogate network.

11. The computer program product of claim 10, wherein computation of the marginal evidence upper bound [symbol] _{MEUBO} is performed by approximating [symbol] _{MEUBO} with
$$\frac{{e}^{- α ξ ( \overline{x} )}}{α {k}_{ψ}} {\sum }_{z ∈ {S}_{ψ}} \frac{{p}^{α} ( z ) {p}_{θ}^{α} ( \tilde{x} \vert z , m )}{{q}_{ψ}^{α - 1} ( z \vert \overline{x} ) \overline{q} ψ ( z \vert \overline{x} )} + ξ ( \overline{x} ) - \frac{1}{α} ,$$
where q̅_{ψ}(z|x̅)=(p(z)+q_{ψ}(z|x̅))/2 and S_{ψ} is Monte-Carlo samples drawn from q̅_{ψ}(z|x̅).

12. The computer program product of claim 8, wherein a discriminative variational autoencoder (DVAE) performs discriminative inference with generative models (DIGM) with the incomplete set of covariates x̅.

13. The computer program product of claim 12, wherein the DVAE includes a generative network, two variational networks, and a surrogate network.

14. The computer program product of claim 13, wherein stochastic gradient-based optimization is employed to minimize the objective function.

15. A computer-implemented method for computing an objective function of discriminative inference with generative models with incomplete data in which some of entries are missing, comprising:
combining a plurality of probability models with a discriminative variational autoencoder (DVAE);
computing a joint evidence lower bound [symbol] _{JELBO} via a first set of the one or more of the plurality of probability models; and
computing a marginal evidence upper bound [symbol] _{MEUBO} via a second set of the one or more of the plurality of probability models.

16. The computer-implemented method of claim 15, wherein the plurality of probability models include a decoder p_{θ}(x̅,y|z), a joint encoder p_{ϕ}(z|x̅,y), and a marginal encoder p_{ψ}(z|x̅).

17. The computer-implemented method of claim 16, wherein the joint evidence lower bound [symbol] _{JELBO} is computed by employing the decoder p_{θ}(x̅,y|z) and the joint encoder p_{ϕ}(z|x̅,y).

18. The computer-implemented method of claim 17, wherein the marginal evidence upper bound [symbol] _{MEUBO} is computed by employing the decoder p_{θ}(x̅,y|z) and the marginal encoder p_{ϕ}(z|x̅,y).

19. The computer-implemented method of claim 15, further comprising computing a predictive distribution [symbol] _{θ}(y|x̅) of an outcome y by using an incomplete set of covariates x̅ and a parameter θ, the parameter θ being unknown.

20. The computer-implemented method of claim 19, further comprising learning the parameter θ by minimizing an objective function [symbol] (θ):=−ln p_{θ}(y|x̅)=ln p_{θ}(\tilde{x}|m)−ln p_{θ}(y,\tilde{x}|m), the objective function [symbol] (θ) bounded with a difference between the marginal evidence upper bound [symbol] _{MEUBO} and the joint evidence lower bound [symbol] _{JELBO}, where ln p_{θ}(\tilde{x}|m)≤ [symbol] _{MEUBO} and ln p_{θ}(y,\tilde{x}|m)≥ [symbol] _{JELBO}.
